(19) United States
(12) Patent Application Publication
(10) Pub. No.: US 2026/0321418 A1
(43) Pub. Date: Oct. 15, 2026

(54) ULTRASONIC DIAGNOSTIC DEVICE, STORING MEDIUM, AND LEARNING MODEL GENERATION METHOD

(71) Applicant: GE Precision Healthcare LLC, Waukesha, WI (US)

(72) Inventor: Naohisa Kamiyama, Hino (JP)

(21) Appl. No.: 19/175,494

(22) Filed: Apr. 10, 2025

(30) Foreign Application Priority Data
Apr. 10, 2025 (JP) 2024-063646

(51) Int. Cl. G16H 50/20; A61B 8/00

(52) U.S. Cl. CPC G16H 50/20; A61B 8/481; A61B 8/5215

(57) ABSTRACT

A method of executing i) a first diagnostic support algorithm that analyzes a first ultrasound image acquired from start of a scan until a first time point has elapsed, and outputs an analysis result, wherein candidates for the analysis result of the first diagnostic support algorithm include a diagnosis result for the subject and information for recommending to a user to continue the ultrasound examination; and ii) a second diagnostic support algorithm that, when the first diagnostic support algorithm outputs information for recommending to the user to continue the ultrasound examination, analyzes a second ultrasound image acquired at a second time point after the first time point and outputs an analysis result, wherein candidates for the analysis result of the second diagnostic support algorithm include a diagnosis result of the subject and information for recommending to the user to continue the ultrasound examination.

### CROSS-REFERENCE TO RELATED APPLICATION

This application claim priority to Japanese Patent Application No. 2024-063646, which was file on Apr. 10, 2024 at the Japanese Patent Office. The entire contents of the above-listed application are incorporated by reference herein in their entirety.

### TECHNICAL FIELD

The present invention relates to an ultrasonic diagnostic device having a diagnostic support function for supporting diagnosis of a subject, a storing medium used in the ultrasonic diagnostic device, and a learning model generation method for generating a learning model that can be used to support the diagnosis.

### BACKGROUND

In the field of medical image diagnosis, a lot of research has been done on image classification using machine learning, and some research has been put to practical use. There are various methods of machine learning, but as an example, “supervised learning” is described below. In supervised learning, medical images (training data) linked to diagnosis results (answers) are prepared, and this training data is then input into machine learning software for learning. A learning model can be generated by this learning. This learning model can be used at a clinical practice when radiologists interpret images using dedicated workstations. Specifically, the radiologist selects an image from a plurality of medical images of a patient that are displayed on the workstation, and inputs the selected image into a learning model, which outputs diagnostic information inferred by machine learning. The diagnostic information output by the learning model can be used as reference information to assist diagnosis.

Furthermore, ultrasonic diagnostic devices are known as devices for non-invasively examining the inside of a body of a subject. An ultrasonic diagnostic device can display cross-sectional images of a living body in real time by an operator pressing an ultrasonic probe against the body surface of a subject (patient) and manipulating the probe.

Furthermore, contrast agents have come to be used in ultrasound examinations, similar to CT and MRI. In a contrast agent examination, a contrast agent is injected into a vein of a subject, and the contrast agent flows through the body, circulating throughout the entire body including an organ of interest. It is expected that ultrasound contrast examinations will provide additional diagnostic information that cannot be determined from cross-sectional images alone, such as the speed at which the contrast agent flows into the area of interest, the pattern of the shape of the area stained by the contrast agent, and patterns depicting changes in staining over time, thereby improving the accuracy of differential diagnosis of benign or malignant tumors. Contrast-enhanced ultrasound examinations are used, for example, for the qualitative diagnosis of breast lesions, for diagnosing the extent of lesions, and the like.

### SUMMARY

A first aspect of the present invention is an ultrasonic diagnostic device that executes a plurality of diagnostic support algorithms to output information necessary to support diagnosis of a subject based on an ultrasound image obtained from a subject to which a contrast agent has been administered, wherein the plurality of diagnostic support algorithms include: a first diagnostic support algorithm that analyzes a first ultrasound image acquired from start of a scan until a first time point has elapsed, and outputs an analysis result, wherein candidates for the analysis result of the first diagnostic support algorithm include a diagnosis result for the subject and information for recommending to a user to continue the ultrasound examination; and a second diagnostic support algorithm that, when the first diagnostic support algorithm outputs information for recommending to a user to continue the ultrasound examination, analyzes a second ultrasound image acquired at a second time point after the first time point and outputs an analysis result, wherein candidates for the analysis result of the second diagnostic support algorithm include a diagnosis result of the subject and information for recommending to a user to continue the ultrasound examination.

The second aspect of the present invention is a computer-executable non-transitory storing medium storing a plurality of diagnostic support algorithms for outputting information necessary to support a diagnosis of a subject based on an ultrasound image obtained from a subject to which a contrast agent has been administered, wherein instructions stored in the storing medium, when executed by the one or more processors, cause the one or more processors to execute the following: a first diagnostic support algorithm that analyzes a first ultrasound image acquired from start of a scan until a first time point has elapsed, and outputs an analysis result, wherein candidates for the analysis result of the first diagnostic support algorithm include a diagnosis result for the subject and information for recommending to a user to continue the ultrasound examination; and a second diagnostic support algorithm that, when the first diagnostic support algorithm outputs information for recommending to a user to continue the ultrasound examination, analyzes a second ultrasound image acquired at a second time point after the first time point and outputs an analysis result, wherein candidates for the analysis result of the second diagnostic support algorithm include a diagnosis result of the subject and information for recommending to a user to continue the ultrasound examination.

A third aspect of the present invention is a method for generating a learning model, comprising: creating a first learning model by having the software learn the first learning data including a first learning image; and creating a second learning model by having the software learn second learning data including a second learning image, the second learning image including a learning image acquired at a time point later than the first learning image.

### BRIEF DESCRIPTION OF THE DRAWINGS

FIG. 1 is a block diagram of an ultrasonic diagnostic device 1 according to an embodiment of the present invention;

FIG. 2 is a diagram depicting an example of flow of a contrast agent examination according to the present embodiment;

FIG. 3 is an explanatory diagram of a method for generating a learning model used in the first diagnostic support algorithm (step ST13);

FIG. 4 is an explanatory diagram of a method for generating a learning model used in the second diagnostic support algorithm (step ST23);

FIG. 5 is an explanatory diagram of a method for generating a learning model used in the i-th diagnostic support algorithm;

FIG. 6 is an explanatory diagram of a method for generating a learning model used in the n-th diagnostic support algorithm, where an example of the flow of step ST3 is depicted in the figure; and

FIG. 7 is a diagram depicting an example of a flow when performing observation from administration of a contrast agent to time point t3.

### DETAILED DESCRIPTION

Although an ultrasonic diagnostic device is operated by a medical technician, a doctor may also operate the ultrasonic diagnostic device. In this case, ultrasound tomographic images can be drawn and diagnosed simultaneously, so the doctor can decide to expand the area to be observed or collect detailed image data over a long period of time, allowing for more detailed observation and diagnosis, which offers great benefits to the patient. On the other hand, a problem with doctors performing ultrasound imaging is the medical cost. Therefore, when performing ultrasound examinations on subjects, doctors must perform medical procedures and manage medical care while taking into consideration the balance between the time required for diagnosis and medical costs.

When considering the balance between the benefits to the patient and medical costs, the fact that a diagnosis using contrast ultrasound takes approximately 10 minutes is one issue that must be addressed. 10 minutes refers to the observation time after administration of the contrast agent. In addition to the observation time, time is required for preliminary confirmation of the tomographic image, securing a venous route, and preparing the contrast agent, so a contrast diagnosis takes about three to five times longer than a simple tomographic image diagnosis. Reducing this time is an important issue.

Therefore, there is a demand for technology that is capable of examining subjects while taking into consideration the balance between the time required for diagnosis and medical costs.

In the present invention, the first diagnostic support algorithm analyzes a first ultrasound image acquired between the start of the scan and a first time point, and outputs an analysis result. A candidate for the analysis results of the first diagnostic support algorithm includes the diagnosis results of the subject.

Therefore, if a diagnosis of the subject is possible based on the first ultrasound image acquired between the start of the scan and the first time point, the first diagnostic support algorithm can output a diagnosis result. Therefore, the first diagnostic support algorithm outputs a diagnosis result for the subject if a diagnosis can be made without ultrasound images from the first time point onwards, allowing the user to avoid a task of obtaining ultrasound images from the second time point onwards that are not necessary for the diagnosis result of the subject.

On the other hand, the candidates for the analysis results of the first diagnostic support algorithm also include information for recommending to the user to continue the ultrasound examination. Therefore, if a diagnosis of the subject cannot be made or if making an accurate diagnosis of the subject is difficult based on only the first ultrasound image acquired between the start of the scan and the first time point, the first diagnostic support algorithm can output information to recommend to the user that the ultrasound examination continue.

Therefore, the first diagnostic support algorithm can determine whether i) a diagnosis result can be output based on the first ultrasound image acquired between the start of the scan and the first time point, or ii) outputting a diagnosis result is difficult from the first ultrasound image, so preferably the user is recommended to continue with the ultrasound examination. Therefore, continuation of the ultrasound examination is only recommended if ultrasound images from the first time point onwards are required for diagnosis, so medical costs incurred by a user (such as a doctor) performing an ultrasound examination can be minimized without reducing diagnostic accuracy.

In addition, when information recommending to the user to continue the ultrasound examination is output, the second diagnostic support algorithm analyzes a second ultrasound image acquired at a second time point after the first time point, and outputs a diagnosis result for the subject. The output candidates of this second diagnostic support algorithm include the diagnosis result of the subject, similarly to the first diagnostic support algorithm.

Therefore, if a diagnosis of the subject is possible based on a second ultrasound image acquired at a second time point after the first time point, the second diagnostic support algorithm can output a diagnosis result. Therefore, the second diagnostic support algorithm outputs a diagnosis result for the subject if a diagnosis can be made without ultrasound images from a time point after the second time point, thereby allowing the user to avoid a task of obtaining ultrasound images from a time point after the third time point that are not required for the diagnosis result for the subject.

On the other hand, the output candidates of the second diagnostic support algorithm also include information for recommending to the user to continue with the ultrasound examination. Therefore, if the second ultrasound image acquired at the second time point alone is not sufficient to diagnose the subject or if accurately diagnosing the subject would be difficult, the second diagnostic support algorithm can output information recommending to the user that the ultrasound examination continue.

Therefore, the second diagnostic support algorithm can determine whether i) a diagnosis result can be output based on the second ultrasound image acquired at the second time point, or ii) that outputting a diagnosis result from the second ultrasound image is difficult, and therefore recommending that the user continue with the ultrasound examination is preferable. Therefore, continuation of the ultrasound examination is only recommended if ultrasound images from a time point later than the second time point are required for diagnosis, thereby minimizing medical costs associated with a user (such as a doctor) performing an ultrasound examination without reducing diagnostic accuracy.

An embodiment for carrying out the invention will be described below, but the present invention is not limited to the following embodiment.

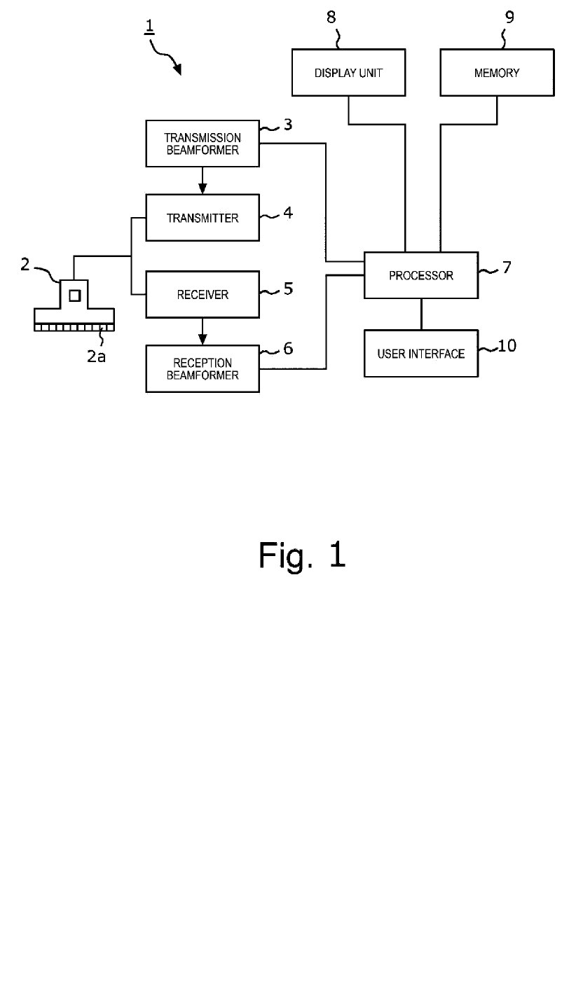

FIG. 1 is a block diagram of an ultrasonic diagnostic device 1 according to an embodiment of the present invention.

The ultrasonic diagnostic device 1 has an ultrasonic probe 2, a transmission beamformer 3, a transmitting apparatus 4, a receiving apparatus 5, a reception beamformer 6, a processor 7, a display unit 8, a memory 9, and a user interface 10.

The ultrasonic probe 2 has a plurality of vibrating elements 2a arranged in an array. The transmission beamformer 3 and the transmitter 4 drive the plurality of vibrating elements 2a, which are arrayed within the ultrasonic probe 2, and ultrasonic waves are transmitted from the vibrating elements 2a. The ultrasonic waves transmitted from the vibrating element 2a are reflected inside the subject, and a reflection echo is received by the vibrating element 2a. The vibrating elements 2a convert the received echo to an electrical signal and output this electrical signal as an echo signal to the receiver 5. The receiver 5 executes a prescribed process on the echo signal and outputs the echo signal to the reception beamformer 6. The reception beamformer 6 executes reception beamforming on the signal received through the receiver 5 and outputs echo data.

The reception beamformer 6 may be a hardware beamformer or a software beamformer. If the reception beamformer 6 is a software beamformer, the reception beamformer 6 may include one or more processors, including one or more of: i) a graphics processing unit (GPU); ii) a microprocessor; iii) a central processing unit (CPU); iv) a digital signal processor (DSP); or v) another type of processor capable of executing logical operations. A processor configuring the reception beamformer 6 may be configured by a processor different from the processor 7 or may be configured by the processor 7.

The ultrasonic probe 2 may include an electrical circuit for performing all or a portion of transmission beamforming and/or reception beamforming. For example, all or a portion of the transmission beamformer 3, the transmitter 4, the receiver 5, and the reception beamformer 6 may be provided in the ultrasonic probe 2.

The processor 7 controls the transmission beamformer 3, the transmitter 4, the receiver 5, and the reception beamformer 6. Furthermore, the processor 7 is in electronic communication with the ultrasonic probe 2. The processor 7 controls which transducer elements 2a are to be activated, and further controls the shape of the ultrasonic beam transmitted from the ultrasonic probe 2. The processor 7 is also in electronic communication with the display 8 and the user interface 10. The processor 7 can process echo data to generate an ultrasound image. The term “electronic communication” may be defined to include both wired and wireless communications. The processor 7 may include a central processing unit (CPU) according to one embodiment. According to another embodiment, the processor 7 may include another electronic component that may perform a processing function such as a digital signal processor, a field programmable gate array (FPGA), a graphics processing unit (GPU), another type of processor, and the like. According to another embodiment, the processor 7 may include a plurality of electronic components capable of executing a processing function. For example, the processor 7 may include two or more electronic components selected from a list of electronic components including a central processing unit, a digital signal processor, a field programmable gate array, and a graphics processing unit.

The processor 7 may also include a complex demodulator (not illustrated in the drawings) that demodulates RF data.

Moreover, the processor 7 may generate various ultrasound images (for example, a B-mode image, color Doppler image, M-mode image, color M-mode image, spectral Doppler image, elastography image, TVI image, strain image, and strain rate image) based on data obtained by processing via the reception beamformer 6. In addition, one or a plurality of modules can generate these ultrasound images.

An image beam and/or an image frame may be saved and timing information may be recorded indicating when the data is retrieved to the memory. The module may include, for example, a scan conversion module that performs a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, and the ultrasound images may be read from the image memory and displayed on the display unit 8.

In the present Specification, the term “image” can broadly indicate both a visual image and data representing a visual image. Furthermore, the term “data” can include raw data, which is ultrasonic data before a scan conversion operation, and image data, which is data after the scan conversion operation.

Note that the processing tasks described above handled by the processor 7 may be executed by a plurality of processors.

Furthermore, when the reception beamformer 6 is a software beamformer, a process executed by the beamformer may be executed by a single processor or may be executed by the plurality of processors.

Examples of the display unit 8 include a LED (Light-Emitting Diode) display, an LCD (Liquid Crystal Display), and an organic EL (Electro-Luminescence) display. The display unit 8 displays an ultrasound image.

The memory 9 is any known data storing medium. In one example, the ultrasonic diagnostic device includes a non-transitory storing medium and a transitory storing medium. In addition, the ultrasonic diagnostic device may also include a plurality of memories. The non-transitory storing medium is, for example, a non-volatile storing medium such as a Hard Disk Drive (HDD) drive, or a Read-Only Memory (ROM), etc. The non-transitory storing medium may include a portable storing medium such as a CD (Compact Disk) or a DVD (Digital Versatile Disk). A program executed by the processor 7 is stored in the non-transitory storing medium. The transitory storing medium is a volatile storing medium such as a Random-Access Memory (RAM).

The memory 9 stores one or more commands that can be executed by the processor 7. The one or a plurality of instructions cause the processor 7 to execute the operations described hereinafter in the embodiments.

Note that the processor 7 may also be configured so as to be able to connect to an external storage device by a wired connection or a wireless connection. In this case, the command causing execution by the processor 7 can be distributed to both the memory 9 and the external storage device for storage.

The user interface 10 can receive input from a user. For example, the user interface 10 receives instruction or information input by the user. The user interface 10 is configured to include a keyboard (keyboard), a hard key (hard key), a trackball (trackball), a rotary control (rotary control), a soft key, and the like. The user interface 10 may include a touch screen that displays a soft key or the like.

The ultrasonic diagnostic device 1 is configured as described above.

Although an ultrasonic diagnostic device 1 is operated by a medical technician, a doctor may also operate the ultrasonic diagnostic device. In this case, ultrasound tomographic images can be drawn and diagnosed simultaneously, so the doctor can decide to expand the area to be observed or collect detailed image data over a long period of time, allowing for more detailed observation and diagnosis, which offers great benefits to the patient. On the other hand, a problem with doctors performing ultrasound imaging is the medical cost. Therefore, when performing ultrasound examinations on subjects, doctors must perform medical procedures while taking into consideration the balance between the time required for diagnosis and medical costs.

Therefore, the inventors of the present application have conducted intensive research and have devised a technique that enables contrast examination of a subject while taking into consideration the balance between the time required for diagnosis and medical costs. The technique is described below.

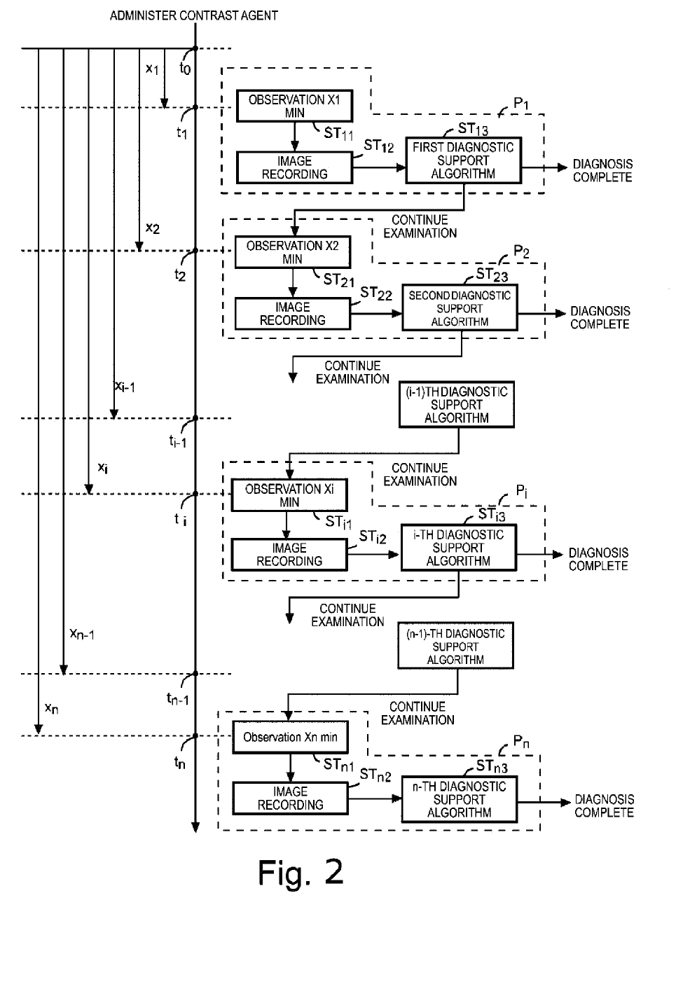

FIG. 2 is a diagram depicting an example of flow of a contrast examination according to the present embodiment.

In this embodiment, the processor is configured to execute a number of diagnostic support algorithms that output information necessary to support the diagnosis of the subject. Contrast examinations are performed using these diagnostic support algorithms. The flow of a contrast examination is described below.

A contrast agent is administered to a subject, and block P1 is executed. Block P1 includes steps ST11 to ST13. On the left side of FIG. 2, a time axis t is depicted, which indicates how much time has passed since the start of the scan. In FIG. 2, the flow of a contrast examination will be described while confirming each time point on the time axis t as necessary.

In step ST11, the user continuously observes the imaging site of the subject and acquires ultrasound images from time point t₀when the scan of the subject is started to a first time point t₁. The acquired ultrasound images may include B-mode images and contrast images. In FIG. 2, the time from time point t₀ to time point t₁ is indicated by X1. The start time point t₀ of the scan may be, for example, a time before administration of the contrast agent is started, a time when administration of the contrast agent is started, a time during which administration of the contrast agent is completed, or a time after administration of the contrast agent is completed.

In step ST12, the user operates the user interface 10 to input a command to record an image. When this command is input, the processor records in the memory 9 the ultrasound images acquired between time point t₀and time point t₁ (time X1).

In step ST13, the processor uses the first diagnostic support algorithm to analyze the ultrasound image recorded in step ST12, and outputs the analysis result. Candidate analysis results output by the first diagnostic support algorithm include the diagnosis results of the subject and information for recommending to the user to continue the ultrasound examination.

If the analysis result in step ST13 indicates a diagnosis (for example, “metastatic tumor”, “cancer”, and the like), the diagnosis is deemed complete and the flow ends.

On the other hand, if it is determined that a diagnosis cannot be made based on the analysis in step ST13 and that further examination of the subject is necessary in order to make a diagnosis, the first diagnostic support algorithm outputs information indicating that continued examination is recommended. If there is a recommendation to continue the examination, the process proceeds to the next block P2. Block P2 includes steps ST21 to ST23.

In step ST21, the user continues observing the imaging site even after X₁ minutes has elapsed since the start of the scan. Specifically, the user observes the imaging site at a second time point t₂ that is later than the first time point t₁ (the time point when X2 minutes have elapsed since the scan start time point t₀), and an ultrasound image is acquired. In step ST21, the user may not only observe at time point t₂, but may also, if necessary, observe the imaging site continuously (or intermittently) from time point t₁ to time point t₂, or may perform observation for only a few seconds before and after time point t₂.

In step ST22, the user operates the user interface 10 to input a command to record an image. When this command is input, the processor records the ultrasound image acquired in step ST21.

In step ST23, the processor uses the second diagnostic support algorithm to analyze the ultrasound image recorded in step ST22, and outputs the analysis result. The second diagnostic support algorithm may analyze not only the ultrasound image recorded in step ST22 but also the ultrasound image recorded in step ST12, and output the analysis results. Candidate analysis results output by the second diagnostic support algorithm include the diagnosis results of the subject and information for recommending to the user to continue the ultrasound examination.

If the analysis result in step ST23 indicates a diagnosis result, the diagnosis is deemed to be complete and the flow ends.

On the other hand, if it is determined that a diagnosis cannot be made based on the analysis in step ST23 and that further examination of the subject is necessary in order to make a diagnosis, the second diagnostic support algorithm outputs information indicating that continued examination is recommended. If there is a recommendation to continue the examination, the process proceeds to the next block.

Similarly, blocks including a step of observing the imaging site, a step of recording an image, and a step of the diagnostic support algorithm are repeatedly executed.

For example, if the (i-1)th diagnostic support algorithm analyzing the ultrasound image acquired at the (i-1)th time point tᵢ₋₁ (where i is an integer greater than or equal to 3) recommends to the user to continue the examination, the process proceeds to block Pi. Block Pi may be executed subsequent to block P2, or block Pi may be executed after one or more other blocks have been executed after block P2. For example, when i=3, that is, when the (i-1)th diagnostic support algorithm is the second diagnostic support algorithm in block P2, if the second diagnostic support algorithm outputs information to recommend to the user to continue with the ultrasound examination, the process proceeds to block Pi. On the other hand, if the second diagnostic support algorithm in block P2 outputs a diagnosis result, the flow ends without proceeding to block Pi.

Block Pi includes steps STi1 to STi3. In step STi1, the user observes the imaging site at the i-th time point tᵢ (the time point when Xi minutes have elapsed since the scan start time point t₀) that is later than the (i-1)-th time point t ᵢ₋₁, and obtains an ultrasound image. Incidentally, in step STi1, the user may not only observe the area at time point tᵢ, but may also observe the area to be imaged continuously (or intermittently) from time point tᵢ₋₁ to time point tᵢ, if necessary, or may observe the area for only a few seconds before and after time point tᵢ.

In step Sti2, the user operates the user interface 10 to input a command to record an image. When this command is input, the processor records the ultrasound image acquired in step Sti1.

In step STi3, the processor uses the i-th diagnostic support algorithm to analyze the ultrasound image recorded in step STi2, and outputs the analysis result. In addition, the i-th diagnostic support algorithm may analyze not only the ultrasound image recorded in step STi2, but also ultrasound images recorded before step STi2 (for example, ultrasound images recorded in step ST12, ultrasound images recorded in step ST22, and the like), and output the analysis results. Candidate analysis results output by the i-th diagnostic support algorithm include the diagnosis results of the subject and information for recommending to the user to continue the ultrasound examination.

If the analysis result in step STi3 indicates a diagnosis result, the diagnosis is deemed to be complete and the flow ends.

On the other hand, if it is determined that a diagnosis cannot be made based on the analysis in step STi3 and that further examination of the subject is necessary in order to make a diagnosis, the i-th diagnostic support algorithm outputs information indicating that continued examination is recommended. If it is recommended to continue the examination, the procedure proceeds to the next block, and similarly, the blocks include steps for observing the imaging site, recording images, and executing the diagnostic support algorithms.

For example, if the (n-1)th diagnostic support algorithm analyzing the ultrasound image acquired at the (n-1)th time point tₙ₋₁ (where n is an integer greater than or equal to i+1) recommends to the user to continue the examination, the process proceeds to block Pn. Block Pn may be executed subsequent to block Pi, or block Pn may be executed after one or more other blocks have been executed after block Pi. For example, when n=i+1, that is, when the (n-1)th diagnostic support algorithm is the i-th diagnostic support algorithm in block Pi, if the i-th diagnostic support algorithm outputs information to recommend to the user to continue with the ultrasound examination, the process proceeds to block Pn.

Block Pn includes steps STn1 to STn3. In step STn1, the user observes the imaging site at the n-th time point tₙ (the time point when Xn minutes have elapsed since the scan start time point t₀) that is later than the (n-1)-th time point t ₙ₋₁, and obtains an ultrasound image. Incidentally, in step STn1, the user may not only observe the area at time point tₙ, but may also observe the area to be imaged continuously (or intermittently) from time point tₙ₋₁ to time point tₙ, if necessary, or may observe the area for only a few seconds before and after time point tₙ.

In step STn2 the user operates the user interface 10 to input a command to record an image. When this command is input, the processor records the ultrasound image acquired in step STn1.

In step STn3, the processor uses the n-th diagnostic support algorithm to analyze the ultrasound image recorded in step STn2, and outputs the analysis result. Candidate analysis results output by the n-th diagnostic support algorithm include the diagnosis results of the subject, but information for recommending to the user to continue the ultrasound examination is not included. In addition, the n-th diagnostic support algorithm may analyze not only the ultrasound image recorded in step STn2, but also ultrasound images recorded before step STn2 (for example, ultrasound images recorded in step ST12, ST22, and STi2), and output the analysis results.

In this manner, the flow for contrast examination ends.

In the flow of FIG. 2, each time an image is recorded, image analysis is executed using a diagnostic support algorithm. If a diagnosis can be made based on the recorded ultrasound images, the diagnosis result is output and the diagnosis is completed. On the other hand, if a diagnosis cannot be made even after analyzing the recorded ultrasound images, information is output to the user recommending that the user continue with the examination. Therefore, the user only needs to continue acquiring ultrasound images if a diagnosis cannot be made using the recorded ultrasound images, thereby minimizing the medical costs associated with the user engaging in ultrasound examinations, without compromising diagnostic accuracy.

Next, a method for performing the diagnostic support algorithm used in the flow of FIG. 2 will be described.

Various techniques have been developed as methods for achieving diagnostic support algorithms, but herein a method that uses a learning model generated by machine learning will be described.

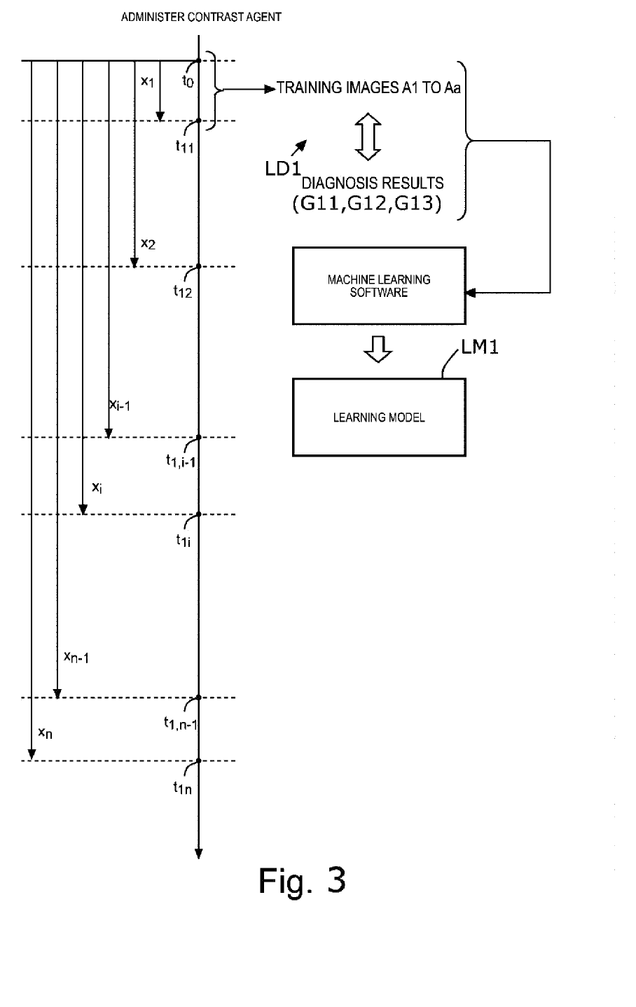

FIG. 3 is an explanatory diagram of a method for generating a learning model used in the first diagnostic support algorithm (step ST13).

Learning data must be prepared when generating a learning model. The learning data can be prepared, for example, by the following procedure.

First, the first learning images A1 to Aa are prepared. The learning images A1 to Aa are a plurality of ultrasound images obtained by actually conducting a contrast examination at a medical facility such as a hospital, and are images acquired during a contrast examination between start time point t₀ of the scan and time point t₁₁ (X₁ minute). Incidentally, the ultrasound images obtained from the medical facilities may be pre-processed as necessary to make images suitable for the learning images A1 to Aa. This pre-processing includes, for example, image cropping, standardization, normalization, image inversion, image rotation, a magnification percentage change, and an image quality change. Furthermore, time point t₁₁ preferably coincides with time point t₁ in the flow of the contrast examination depicted in FIG. 2. However, time point t₁₁ is not required to strictly coincide with time point t₁, and time point t₁₁ may be a certain time difference from time point t₁as long as the reliability of the analysis results of the first diagnostic support algorithm (step ST13) can be sufficiently ensured.

Next, the diagnosis results are linked to the learning images A1 to Aa as correct answer data. For example, if the diagnosis of image A1 is a metastatic tumor, image A1 is linked to the diagnosis of “metastatic tumor”, and if the diagnosis of image A2 is cancer, image A2 is linked to the diagnosis of “cancer”. Here, for ease of description, it is assumed that there are three diagnosis results (G11, G12, G13) as correct answer data, and each of the learning images A1 to Aa is linked to one of the three diagnosis results.

The learning data LD1 can be prepared in this manner.

Next, the learning data LD1 can be input to machine learning software and trained to generate a learning model LM1 used in the first diagnostic support algorithm.

When an image with an unknown diagnosis result is input, learning model LM1 is trained to predict the probability that the input image corresponds to diagnosis result G11, the probability that the image corresponds to diagnosis result G12, and the probability that the image corresponds to diagnosis result G13, and to output the diagnosis result with the highest probability as the analysis result. However, even if the diagnosis result has the highest probability, if the probability has a low value, there is a risk that the diagnosis result obtained is not highly reliable. For example, if the probability of diagnosis result G11 is 30%, the probability of diagnosis result G12 is 30%, and the probability of diagnosis result G13 is 30%, then all of the diagnosis results are considered to be unreliable. In this case, outputting a highly reliable diagnosis result using only the images acquired between time point t₀ and time point t₁₁will be difficult. Therefore, the learning model LM1 sets a threshold value TH for the probability of a diagnosis result, and is trained to recommend to the user to continue the ultrasound examination if the probability of any diagnosis result does not exceed the threshold value TH. Therefore, continuation of the ultrasound examination is only recommended if subsequent ultrasound images are required for diagnosis, so medical costs incurred by a user (such as a doctor) performing an ultrasound examination can be minimized without reducing diagnostic accuracy.

In the above example, three diagnosis results (G11, G12, G13) were used as correct answer data, but the diagnosis results used as correct answer data are not limited to three, and one or two diagnosis results may be used as correct answer data, or four or more diagnosis results may be used as correct answer data. For example, if only one diagnosis result G11 is used as the correct data, when an image with an unknown diagnosis result is input, the learning model LM1 predicts the probability that the input image corresponds to the diagnosis result G11, and if the predicted probability does not exceed the threshold value TH, information recommending that the user continue with the ultrasound examination can be output.

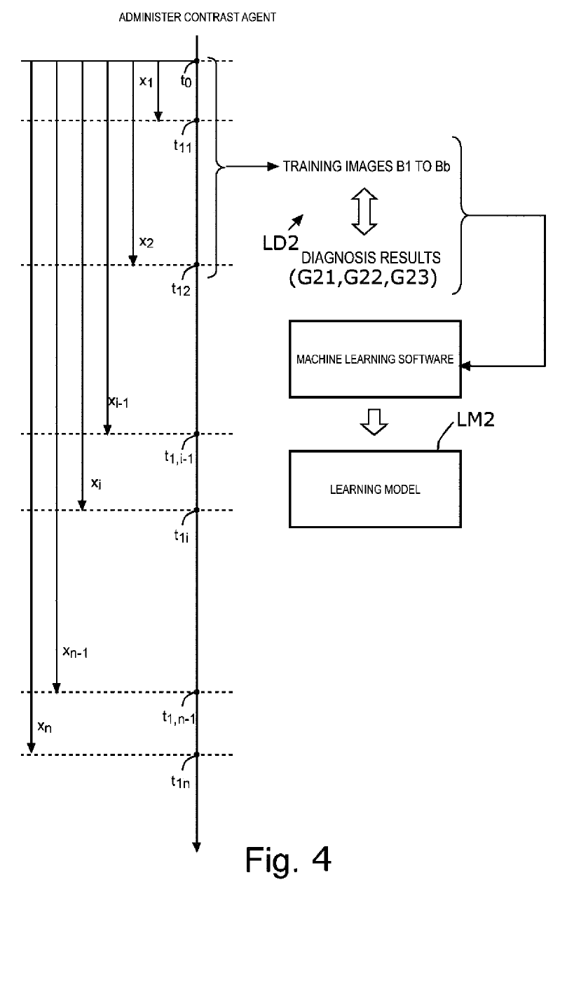

FIG. 4 is an explanatory diagram of a method for generating a learning model used in the second diagnostic support algorithm (step ST23).

First, the second learning images B1 to Bb are prepared. The learning images B1 to Bb are a plurality of ultrasound images obtained by actually conducting a contrast examination at a medical facility such as a hospital, and are images acquired during a contrast examination between start time point t₀ of the scan and time point t₁₂ (X₂ minutes). Therefore, the training images B1 to Bb include training images acquired at a later time point than the training images A1 to Aa (see FIG. 3) acquired between time point t₀ and time point t₁₁. Incidentally, the ultrasound images obtained from the medical facilities may be pre-processed as necessary to make images suitable for the learning images B1 to Bb. Furthermore, time point t₁₂ preferably coincides with time point t2 in the flow of the contrast examination depicted in FIG. 2. However, time point t₁₂ is not required to strictly coincide with time point t₂, and time point t₁₂ may be a certain time difference from time point t₂ as long as the reliability of the analysis results of the second diagnostic support algorithm (step ST23) can be sufficiently ensured. Note that in this embodiment, the training images B1 to Bb are ultrasound images acquired between time point t₀ and time point t₁₂, but may include only the ultrasound image at time point t₁₂ as long as the reliability of the analysis results of the second diagnostic support algorithm (step ST23) can be sufficiently ensured. Furthermore, as long as the learning images B1 to Bb include an ultrasound image at time point t₁₂, ultrasound images acquired continuously (or intermittently) between the scan start time point t₀ and time point t₁₂may be included, or ultrasound images acquired within a few seconds before or after time point t₁₂ may be included.

Next, the diagnosis results are linked to the learning images B1 to Bb as correct answer data. Here, for ease of description, it is assumed that there are three diagnosis results (G21, G22, G23) as correct answer data, and each of the learning images B1 to Bb is linked to one of the three diagnosis results (G21, G22, G23).

In this manner, the learning data LD2 can be prepared.

Next, the learning data LD2 is input into machine learning software and trained. In this manner, the learning model LM2 used in the diagnostic support algorithm 12 can be generated.

Incidentally, when an image with an unknown diagnosis result is input, the learning model LM2 is trained to predict the probability that the input image corresponds to diagnosis result G21, the probability of corresponding to diagnosis result G22, and the probability of corresponding to diagnosis result G23, and to output the diagnosis result with the highest probability as the analysis result. However, even if the diagnosis result has the highest probability, if the probability has a low value, there is a risk that the diagnosis result obtained is not highly reliable. For example, if the probability of diagnosis result G21 is 30%, the probability of diagnosis result G22 is 30%, and the probability of diagnosis result G23 is 30%, then all of the diagnosis results are considered to be unreliable. In this case, outputting a highly reliable diagnosis result using only the images acquired between time point t₀ and time point t12 will be difficult. Therefore, the learning model LM2 sets a threshold value TH for the probability of a diagnosis result, and is trained to recommend to the user to continue the ultrasound examination if the probability of any diagnosis result does not exceed the threshold value TH. Therefore, continuation of the ultrasound examination is only recommended if subsequent ultrasound images are required for diagnosis, so medical costs incurred by a user (such as a doctor) performing an ultrasound examination can be minimized without reducing diagnostic accuracy.

In the above example, three diagnosis results (G21, G22, G23) were used as correct answer data, but the diagnosis results used as correct answer data are not limited to three, and one or two diagnosis results may be used as correct answer data, or four or more diagnosis results may be used as correct answer data. For example, if only one diagnosis result G21 is used as the correct data, when an image with an unknown diagnosis result is input, the learning model LM2 predicts the probability that the input image corresponds to the diagnosis result G21, and if the predicted probability does not exceed the threshold value TH, information recommending that the user continue with the ultrasound examination can be output.

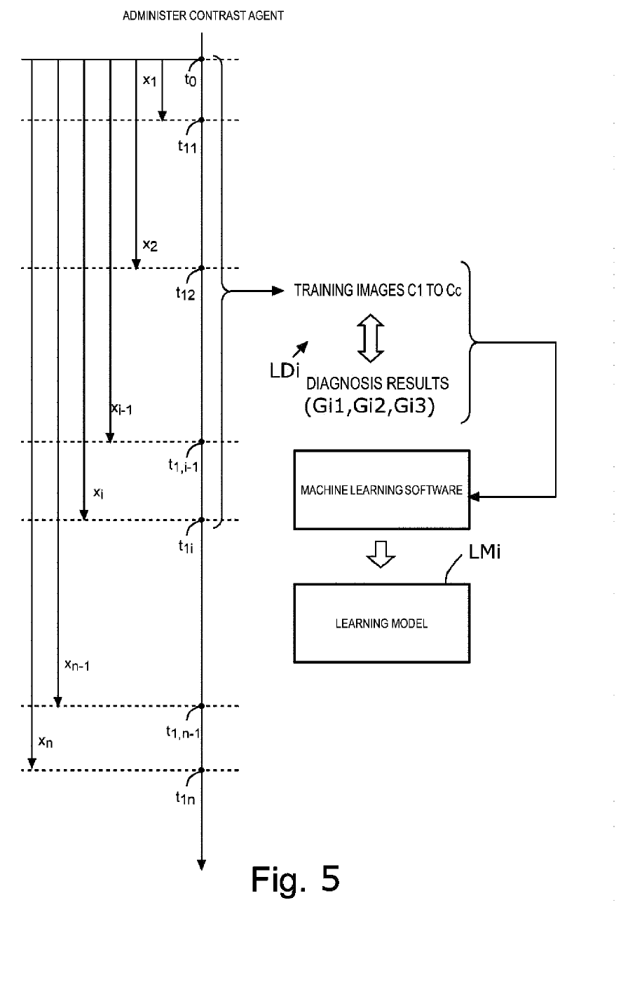

Similarly, a learning model can be generated for use in each diagnostic support algorithm. For example, when generating a learning model to be used in the i-th diagnostic support algorithm in step STi3, the i-th learning images C1 to Cc are prepared as depicted in FIG. 5. The learning images C1 to Cc are a plurality of ultrasound images obtained by actually conducting a contrast examination at a medical facility such as a hospital, and are images acquired during a contrast examination between start time point t₀ of the scan and time point t₁ᵢ (Xᵢ minutes). Therefore, the training images C1 to Cc include training images captured at a time point t₁ᵢ that is later than the time range t₀ to t₁₂ (see FIG. 4) during which the training images B1 to Bb were captured. Incidentally, the ultrasound images obtained from the medical facilities may be pre-processed as necessary to make images suitable for the learning images C1 to Cc. Furthermore, time point t₁ᵢ preferably coincides with time point tᵢ in the flow of the contrast examination depicted in FIG. 2. However, time point t₁ᵢis not required to strictly coincide with time point tᵢ, and time point t₁ᵢ may be a certain time difference from time point tᵢ as long as the reliability of the analysis results of the i-th diagnostic support algorithm (step STi3) can be sufficiently ensured. Note that in this embodiment, the training images C1 to Cc are ultrasound images acquired between time point t₀ and time point t₁ᵢ, but may include only the ultrasound image at time point t₁ᵢas long as the reliability of the analysis results of the second diagnostic support algorithm (step STi3) can be sufficiently ensured. Furthermore, as long as the learning images C1 to Cc include an ultrasound image at time point t₁ᵢ, ultrasound images acquired continuously (or intermittently) between the scan start time point t₀and time point t₁ᵢ may be included, or ultrasound images acquired within a few seconds before or after time point t₁ᵢ may be included. Once the learning images C1 to Cc are prepared, the i-th learning data LDi is prepared by linking the images to the correct answer data as described above. Next, the i-th learning model LMi can be generated by executing the learning process.

In addition, similar to learning models LM1 and LM2, learning model LMi sets a threshold value TH for the probability of diagnosis results, and learns to recommend to the user to continue the ultrasound examination if the probability of all diagnosis results do not exceed the threshold TH. Therefore, continuation of the ultrasound examination is only recommended if subsequent ultrasound images are required for diagnosis, so medical costs incurred by a user (such as a doctor) performing an ultrasound examination can be minimized without reducing diagnostic accuracy.

In the above example, three diagnosis results (Gi1, Gi2, Gi3) were used as correct answer data, but the diagnosis results used as correct answer data are not limited to three, and one or two diagnosis results may be used as correct answer data, or four or more diagnosis results may be used as correct answer data. For example, if only one diagnosis result Gi1 is used as the correct data, when an image with an unknown diagnosis result is input, the learning model LMi predicts the probability that the input image corresponds to the diagnosis result Gi1, and if the predicted probability does not exceed the threshold value TH, information recommending that the user continue with the ultrasound examination can be output.

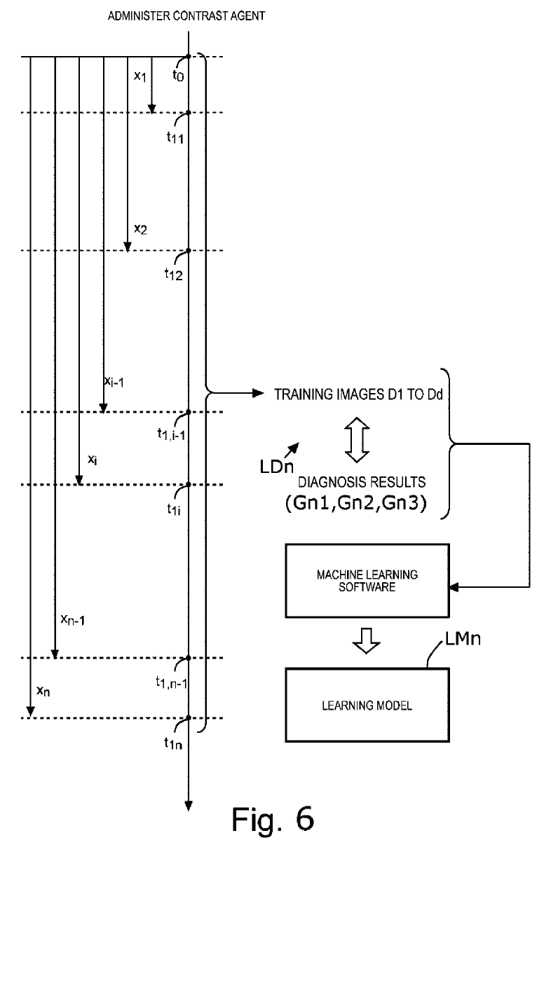

Furthermore, when generating a learning model to be used in the n-th diagnostic support algorithm in step STn3, the n-th learning images D1 to Dd are prepared as depicted in FIG. 6. The learning images D1 to Dd are a plurality of ultrasound images obtained by actually conducting a contrast examination at a medical facility such as a hospital, and are images acquired during a contrast examination between start time point t₀ of the scan and time point t₁ₙ (Xₙ minutes). Therefore, the training images D1 to Dd include training images captured at time point t₁ₙ that is later than the time range of t₀ to t₁ᵢ (see FIG. 5) during which the training images C1 to Cc were captured. Incidentally, the ultrasound images obtained from the medical facilities may be pre-processed as necessary to make images suitable for the learning images D1 to Dd. Furthermore, time point t₁ₙ preferably coincides with time point tₙ in the flow of the contrast examination depicted in FIG. 2. However, time point t₁ₙ is not required to strictly coincide with time point tₙ, and time point t₁ₙ may be a certain time difference from time point tₙ as long as the reliability of the analysis results of the n-th diagnostic support algorithm (step STn3) can be sufficiently ensured. In this embodiment, the training images D1 to Dd are ultrasound images acquired between time t₀and time t₁ₙ, but may include only the ultrasound image at time t₁ₙ as long as the reliability of the analysis results of the nth diagnostic support algorithm (step STn3) can be sufficiently ensured. Furthermore, as long as the learning images D1 to Dd include an ultrasound image at time point t₁ₙ, ultrasound images acquired continuously (or intermittently) between the scan start time point t₀ and time point t₁ₙmay be included, or ultrasound images acquired within a few seconds before or after time point t₁ₙ may be included. Once the learning images D1 to Dd are prepared, the nth learning data LDn is prepared by linking the images with the correct data, and the learning process is executed to generate the nth learning model LMn.

The learning model LMn executes analysis of the ultrasound image acquired in the final observation (step STn1) of the contrast examination. Therefore, ultrasound examinations are not expected to be continued after analysis using the learning model LMn. Therefore, compared to the learning models LM1, LM2, and LMi described above, the learning model LMn differs in that there is no training to recommend to the user that the ultrasound examination should be continued.

In the present embodiment, the learning models LD1 to LDn were generated as described above. The learning models LD1 to LDn output a diagnosis result if a diagnosis is possible from the acquired ultrasound image, but on the other hand, if a diagnosis is not possible from the acquired ultrasound image, the learning models recommend to the user to continue with the ultrasound examination. Therefore, continuation of the ultrasound examination is only recommended if subsequent ultrasound images are required for diagnosis, so medical costs incurred by a user (such as a doctor) performing an ultrasound examination can be minimized without reducing diagnostic accuracy.

Next, as an example of the flow depicted in FIG. 2, a flow in which n=3, that is, observation is performed from administration of a contrast agent until time point t₃, will be described.

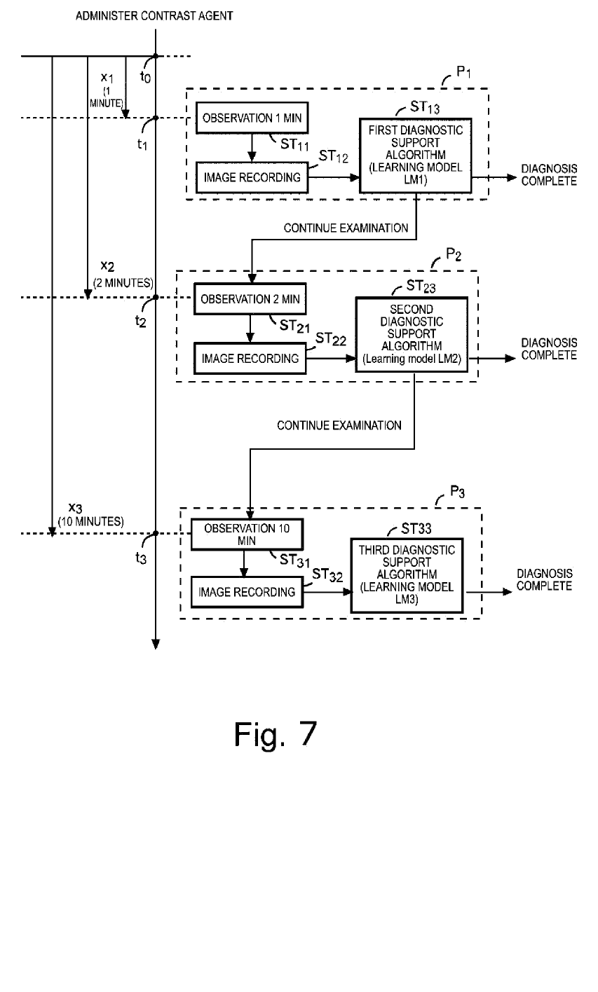

FIG. 7 is a diagram depicting an example of a flow when performing observation from administration of a contrast agent to time point t₃.

After administering the contrast agent, in step ST11, the user continuously observes the imaging site of the subject and acquires ultrasound images from time point t₀ when the scan of the subject begins to time point t₁ (time X1). The acquired ultrasound images may include B-mode images and contrast images. The time X1 can be, for example, X1=1 minute. In step ST12, the image acquired in step ST11 is recorded. Then, in step ST13, the processor inputs the recorded ultrasound image (or the ultrasound image that has been preprocessed as necessary) as an input image to the learning model LM1 of the first diagnostic support algorithm. The learning model LM1 analyzes the input image, and if a diagnosis can be made based on the input image alone, the learning model outputs a diagnosis result. On the other hand, if a diagnosis cannot be made based on the input image alone, the learning model LM1 outputs information indicating that continued examination is recommended. This information is displayed, for example, on a display unit 8 (see FIG. 1). If information recommending continuation of the examination is displayed on the display unit 8, the process proceeds to step ST21, where the user continues the examination.

In step ST21, the user observes the imaging site from the first time point t₁ to the second time point t₂ and obtains ultrasound images. A second time point t₂ indicates the time X2 that has elapsed since the scan start time point t₀. The time X2 may be, for example, X2=2 minutes. In step ST21, the user might only observe at time point t₂, but may also, if necessary, observe the imaging site continuously (or intermittently) from time point t₁to time point t₂, or may perform observation for only a few seconds before and after time point t₂. In step ST22, the image acquired in step ST21 is recorded. Then, in step ST23, the processor inputs the recorded ultrasound image (or the ultrasound image that has been preprocessed as necessary) as an input image to the learning model LM2 of the second diagnostic support algorithm. The learning model LM2 analyzes the input image, and if a diagnosis can be made based on the input image alone, the learning model outputs a diagnosis result. In addition, the learning model LM2 may analyze not only the ultrasound image recorded in step ST22, but also ultrasound images recorded before step ST22 (for example, ultrasound images recorded in step ST12), and output the analysis results. If a diagnosis cannot be made based on the input image alone, the learning model LM2 outputs information indicating that continued examination is recommended. If this information is output, the process proceeds to step ST31, and the user continues the examination.

In step ST31, the user observes the imaging site from the second time point t₂ to the third time point t₃ and obtains ultrasound images. A third time point t₃ indicates the time X3 that has elapsed since the scan start time point t₀. The time X3 may be, for example, X3=10 minutes. In step ST31, the user might only observe at time point t₃, but may also, if necessary, observe the imaging site continuously (or intermittently) from time point t₂ to time point t₃, or may perform observation for only a few seconds before and after time point t₃. In step ST32, the image acquired in step ST31 is recorded. Furthermore, in step ST33, the processor inputs the recorded ultrasound image (or the ultrasound image that has been preprocessed as necessary) as an input image to the learning model LM3 of the third diagnostic support algorithm. The learning model LM3 analyzes the input image and outputs a diagnosis result. In addition, the learning model LM3 may analyze not only the ultrasound image recorded in step ST32, but also ultrasound images recorded before step ST32 (for example, ultrasound images recorded in step ST12, ultrasound images recorded in step ST22, and the like), and output the diagnosis results.

In this manner, the diagnosis is completed.

As described above, in the present embodiment, if a diagnosis is possible from the acquired ultrasound image, a diagnosis result is output, but if a diagnosis is not possible from the acquired ultrasound image, the user is recommended to continue with the ultrasound examination. Therefore, continuation of the ultrasound examination is recommended only when further ultrasound images are required for diagnosis, eliminating the need to require 10 minutes for every contrast examination. As a result, detailed diagnostic information can be obtained while ensuring the time efficiency of the contrast examination.

In the present embodiment, the diagnostic support algorithm uses machine learning technology to output a diagnosis result (or information recommending continued examination). However, in the present invention, the diagnostic support algorithm is not limited to one that utilizes AI technology such as machine learning, and a diagnosis result (or information recommending continued examination) may be output using an algorithm that does not use AI technology. For example, a region of interest may be set in an ultrasound image, the pixel values of pixels within the region of interest may be compared with the pixel values of pixels in a region surrounding the region of interest, and a diagnosis result (or information recommending continued examination) may be output based on the comparison result.

What is claimed is:

1. An ultrasonic diagnostic device, comprising:
a memory storing instructions; and
a processor configured to execute the instructions to execute a plurality of diagnostic support algorithms,
wherein the plurality of diagnostic support algorithms comprise:
a first diagnostic support algorithm that analyzes a first ultrasound image acquired from start of a scan until a first time point has elapsed, and outputs an analysis result, wherein candidates for the analysis result of the first diagnostic support algorithm include a diagnosis result for the subject and information for recommending to a user to continue the ultrasound examination; and
a second diagnostic support algorithm that, when the first diagnostic support algorithm outputs information for recommending to the user to continue the ultrasound examination, analyzes a second ultrasound image acquired at a second time point after the first time point and outputs an analysis result, wherein candidates for the analysis result of the second diagnostic support algorithm include a diagnosis result of the subject and information for recommending to the user to continue the ultrasound examination.

2. The ultrasonic diagnostic device according to claim 1, wherein
the plurality of diagnostic support algorithms include:
a third diagnostic support algorithm that, when the second diagnostic support algorithm outputs information for recommending to the user to continue the ultrasound examination, analyzes an third ultrasound image acquired after the second ultrasound image, wherein candidates for the analysis result of the third diagnostic support algorithm include a diagnosis result of the subject and information for recommending to the user to continue the ultrasound examination.

3. The ultrasonic diagnostic device according to claim 2, wherein
the first diagnostic support algorithm includes a first learning model generated based on first learning data including a first learning image;
the second diagnostic support algorithm includes a second learning model generated based on second learning data including a second learning image; and
the second learning image includes a learning image acquired at a later time point than the first learning image.

4. The ultrasonic diagnostic device according to claim 3, wherein the third diagnostic support algorithm includes a third learning model generated based on third learning data including a third learning image; and
the third learning image includes a learning image acquired at a later time point than the second learning image.

5. The ultrasonic diagnostic device according to claim 4, wherein
one diagnosis result is included in a candidate analysis result output by the first learning model, the second learning model, or the third learning model; and
the first learning model, the second learning model, or the third learning model, when an image is input, predicts the probability that the input image corresponds to one of the diagnosis results, and when the predicted probability does not exceed a threshold, outputs information recommending to the user to continue the ultrasound examination.

6. The ultrasonic diagnostic device according to claim 4, wherein
a plurality of diagnosis results are included in a candidate analysis result output by the first learning model, the second learning model, or the third learning model; and
the first learning model, the second learning model, or the third learning model, when an image is input to the second learning model, predicts the probability that the input image corresponds to each of the diagnosis results, and when the probability that all of the diagnosis results do not exceed a threshold, outputs information recommending to the user to continue the ultrasound examination.

7. The ultrasonic diagnostic device according to claim 1, wherein
the plurality of diagnostic support algorithms include:
a third diagnostic support algorithm that, when the second diagnostic support algorithm outputs information for recommending to the user to continue the ultrasound examination, analyzes a third ultrasound image acquired at a third time point after the second time point and outputs an analysis result, wherein candidates for the analysis result of the third diagnostic support algorithm include a diagnosis result of the subject.

8. The ultrasonic diagnostic device according to claim 1, wherein the second diagnostic support algorithm analyzes the first ultrasound image in addition to the second ultrasound image.

9. The ultrasonic diagnostic device according to claim 2, wherein the third diagnostic support algorithm analyzes an ultrasound image acquired prior to the third ultrasound image in addition to the third ultrasound image.

10. The ultrasonic diagnostic device according to claim 1, wherein the first ultrasound image includes a B-mode image and a contrast image.

11. The ultrasonic diagnostic device according to claim 1, wherein the first diagnostic support algorithm analyzes the first ultrasound image based on pixel values of pixels within a region of interest of the first ultrasound image and pixel values of pixels in a region surrounding the region of interest.

12. The ultrasonic diagnostic device according to claim 1, wherein the second diagnostic support algorithm analyzes the second ultrasound image based on pixel values of pixels within a region of interest of the second ultrasound image and pixel values of pixels in a region surrounding the region of interest.

13. A non-transitory computer readable storage medium storing a plurality of instruction that, when executed by a computer, cause the computer to execute a plurality of diagnostic support algorithms,
wherein the plurality of diagnostic support algorithms comprise:
a first diagnostic support algorithm that analyzes a first ultrasound image acquired from start of a scan until a first time point has elapsed, and outputs an analysis result, wherein candidates for the analysis result of the first diagnostic support algorithm include a diagnosis result for the subject and information for recommending to a user to continue the ultrasound examination; and
a second diagnostic support algorithm that, when the first diagnostic support algorithm outputs information for recommending to the user to continue the ultrasound examination, analyzes a second ultrasound image acquired at a second time point after the first time point and outputs an analysis result, wherein candidates for the analysis result of the second diagnostic support algorithm include a diagnosis result of the subject and information for recommending to the user to continue the ultrasound examination.

14. The non-transitory computer readable storage medium according to claim 13, wherein
the plurality of diagnostic support algorithms include:
a third diagnostic support algorithm that, when the second diagnostic support algorithm outputs information for recommending to the user to continue the ultrasound examination, analyzes an third ultrasound image acquired after the second ultrasound image, wherein candidates for the analysis result of the third diagnostic support algorithm include a diagnosis result of the subject and information for recommending to the user to continue the ultrasound examination.

15. The non-transitory computer readable storage medium according to claim 14, wherein
the first diagnostic support algorithm includes a first learning model generated based on first learning data including a first learning image;
the second diagnostic support algorithm includes a second learning model generated based on second learning data including a second learning image; and
the second learning image includes a learning image acquired at a later time point than the first learning image.

16. The non-transitory computer readable storage medium according to claim 15, wherein the third diagnostic support algorithm includes a third learning model generated based on third learning data including a third learning image; and
the third learning image includes a learning image acquired at a later time point than the second learning image.

17. The non-transitory computer readable storage medium according to claim 16, wherein
one diagnosis result is included in a candidate analysis result output by the first learning model, the second learning model, or the third learning model; and
the first learning model, the second learning model, or the third learning model, when an image is input, predicts the probability that the input image corresponds to one of the diagnosis results, and when the predicted probability does not exceed a threshold, outputs information recommending to the user to continue the ultrasound examination.

18. The non-transitory computer readable storage medium according to claim 16, wherein
a plurality of diagnosis results are included in a candidate analysis result output by the first learning model, the second learning model, or the third learning model; and
the first learning model, the second learning model, or the third learning model, when an image is input to the second learning model, predicts the probability that the input image corresponds to each of the diagnosis results, and when the probability that all of the diagnosis results do not exceed a threshold, outputs information recommending to the user to continue the ultrasound examination.

19. The non-transitory computer readable storage medium according to claim 13, wherein
the plurality of diagnostic support algorithms include:
a third diagnostic support algorithm that, when the second diagnostic support algorithm outputs information for recommending to the user to continue the ultrasound examination, analyzes a third ultrasound image acquired at a third time point after the second time point and outputs an analysis result, wherein candidates for the analysis result of the third diagnostic support algorithm include a diagnosis result of the subject.

20. A method of executing a plurality of diagnostic support algorithms,
wherein the plurality of diagnostic support algorithms comprise:
a first diagnostic support algorithm that analyzes a first ultrasound image acquired from start of a scan until a first time point has elapsed, and outputs an analysis result, wherein candidates for the analysis result of the first diagnostic support algorithm include a diagnosis result for the subject and information for recommending to a user to continue the ultrasound examination; and
a second diagnostic support algorithm that, when the first diagnostic support algorithm outputs information for recommending to the user to continue the ultrasound examination, analyzes a second ultrasound image acquired at a second time point after the first time point and outputs an analysis result, wherein candidates for the analysis result of the second diagnostic support algorithm include a diagnosis result of the subject and information for recommending to the user to continue the ultrasound examination.
